(19) United States
(12) Patent Application Publication
(10) Pub. No.: US 2005/0096371 A1
(43) Pub. Date: May 5, 2005

(54) Topical pharmaceutical compositions

(76) Inventors: Anandi Krishnan, Navi Mumbai (IN); Nilendu Sen, Navi Mumbai (IN); Kavita Chandurkar, St. John's NL (CA)

Correspondence Address: DILWORTH & BARRESE, LLP, 333 EARLE OVINGTON BLVD., UNIONDALE, NY, 11553, US

(21) Appl. No.: 10/982,980

(22) Filed: Nov. 5, 2004

Related U.S. Application Data
(60) Provisional application No. 60/517,588, filed on Nov. 5, 2003.

(51) Int. Cl. A61K 31/415

(52) U.S. Cl. 514/406

(57) ABSTRACT

A hydroalcoholic topical pharmaceutical composition is provided comprising a therapeutically effective amount of a therapeutic agent comprising one or more selective cyclooxygenase-2 (COX-2) inhibitors or pharmaceutically acceptable salts or esters thereof solubilized in a solubilizing amount of a penetration vehicle system comprising a skin penetration enhancing effective amount of at least one monohydric alcohol and at least two non-volatile organic compounds selected from the group consisting of pyrrolidones, polyol ethers, polyols and mixtures thereof. Also provided is a process for its preparation.

### CROSS REFERENCE TO RELATED APPLICATIONS

This application claims the benefit under 35 U.S.C. § 119 to U.S. Provisional Application No. 60/517,588, filed Nov. 5, 2003, the contents of which are incorporated by reference herein.

### BACKGROUND OF THE INVENTION

1. Technical Field

The present invention relates generally to an anti-inflammatory and/or analgesic topical pharmaceutical compositions and a process for preparing same.

2. Description of the Related Art

The administration of drugs and other biological materials to the bloodstream via a transdermal route or to the localized site of action has received much attention in recent years. The skin of an average adult covers more than two square meters of surface area and receives about one-third of all blood circulating through the body. It is elastic, rugged, and generally self-generating. The skin generally consists of three layers: the stratum corneum, the epidermis, and the dermis. The stratum corneum represents the rate-limiting step in diffusion of chemicals through the skin. The stratum corneum is composed of dead, keratinized, metabolically inactive cells, which are closely packed together, and consists of an amorphous matrix of mainly lipoid and nonfibrous protein within which keratin filaments are distributed. The cells of the stratum corneum generally contain 20% water, while the cells below, in the stratum germinativum contain 70% water. The stratum corneum does not become hydrated readily. Thus, transdermal permeation is primarily controlled by diffusion through the stratum corneum.

Due to the availability of large surface area, easy accessibility, application dynamics and the noninvasive nature of the therapy, topical administration of drugs has long been considered a promising route of drug delivery whether the bioavailability desired is systemic, dermal, regional or localized. This mode of drug delivery provides many advantages over customarily used routes of administration. For example, it bypasses the portal circulation and thereby the hepatic first-pass metabolism, avoids the variable systemic absorption and metabolism and also, potentially reduces gastrointestinal irritation associated with oral administration. Additionally, it avoids the risks and patient noncompliance associated with parenteral treatment. Topical route offers continuity of drug administration, permits use of therapeutic agents with short biological half-lives, provides treatment of cutaneous manifestations of diseases usually treated systemically delivers medication directly into the systemic circulation and fosters ease of use and total patient compliance.

Nonsteroidal anti-inflammatory drugs (“NSAIDs”) are a group of structurally diverse compounds used clinically for the successful treatment of a range of disorders that are associated with pain and/or inflammation (including arthritic disorders). NSAIDs are known to inhibit the cyclooxygenase (“COX”) enzymes, which catalyze the conversion of arachidonic acid to the various prostaglandins, and the drugs are believed to exert their analgesic and anti-inflammatory effects through inhibition of the COX enzymes. Two isoforms of the COX enzyme have been identified in eukaryotic cells, cyclooxygenase-1 (“COX-1”) and cyclooxygenase-2 (“COX-2”). The COX-1 protein is constitutively expressed (i.e., it is present under normal conditions and does not need to be induced) and is involved in the maintenance of homeostatic conditions. For example, COX-1 plays a role in blood clotting and elicits a protective role in organs such as the gastrointestinal tract. The COX-2 protein, on the other hand, is inducible and is involved in the immediate early gene response to various stimuli such as cytokines, growth factors and UV light. Older NSAIDs such as, for example, aspirin, ibuprofen and flurbiprofen, inhibit both forms of COX and are referred to as non-selective NSAIDs. Newer NSAIDs such as, for example, celecoxib and rofexocib, are selective for COX-2 and are therefore referred to as selective COX-2 inhibitors.

U.S. Pat. No. 5,093,133 discloses a method for the topical delivery of the drug ibuprofen through the skin in order to treat conditions in the joints or soft tissue beneath the skin (e.g., inflammation and/or pain) and more particularly, a percutaneous delivery system wherein ibuprofen, preferably as a substantially pure S-enantiomer is incorporated into a hydroalcoholic gel having a pH of 3.5 to 6.0.

U.S. Pat. No. 5,976,566 discloses an alcoholic or aqueous alcoholic composition which comprises of from about 2-10% ibuprofen, a skin penetration enhancer of from about 4-15% of a C₇ to C₁₄ hydrocarbyl substituted 1,3-dioxolane or acetal, 0-18% of glycol having from 3-6 carbon atoms, at least about 40% of volatile alcohol selected from the group consisting of ethanol, isopropanol and mixture thereof, 0-25% water, and base to provide a pH in the range of from 6.5 to 8.

U.S. Pat. No. 4,393,076 discloses an anti-inflammatory and analgesic gel composition comprising, by weight, (a) 0.5 to 10% of ketoprofen as the effective ingredient, (b) 2 to 40% of a glycol selected from the group consisting of propylene glycol, butylene glycol, polyethylene glycol, polypropylene glycol, polyethylene glycol dodecyl ether and glycerine, (c) 20 to 55% of water, (d) up to 60% of a lower alcohol selected from the group consisting of ethanol, denatured ethanol, propanol and isopropanol, (e) 0.5 to 5% of a gelling agent selected from the group consisting of carboxyvinyl polymers, hydroxycellulose, methylcellulose, carboxymethylcellulose, hydroxypropylcellulose and alginic acid-propylene glycol ester and (e) 0.1 to 3% of a neutralizing agent selected from the group consisting of triethanolamine, diethylamine, triethylamine, diisopropylamine and diisopropanolamine. Ketoprofen belongs to the propionic acid class of NSAIDs, which are non-selective cyclooxygenase inhibitors.

U.S. Pat. No. 5,716,609 discloses a therapeutic anti-inflammatory analgesic pharmaceutical composition containing nimesulide for use transdermally. The pharmaceutical composition comprises nimesulide, one or more vehicles/bases and water. The compositions may also contain one or more surfactants and a neutralizing/pH adjusting agent. Nimesulide is a therapeutic anti-inflammatory agent, which is a non-selective cyclooxygenase inhibitor.

WO Patent No. 0303580 discloses a pharmaceutical composition for topical delivery comprising a pharmaceutically effective amount of a drug that acts selectively as a COX-2 inhibitor, wherein the drug has a mean particle size of less than about 30 micron, a gelling agent and a solubilizing agent. The drug is dispersed as micronized, undissolved particles in the gel base. The publication discloses preparations containing celecoxib and rofecoxib.

U.S. Pat. No. 5,633,272 discloses administering valdecoxib, 4-[5-methyl-3-phenyl-isoxazol-4-yl] benzenesulfonamide, intravascularly, intraperitoneally, subcutaneously, intramuscularly or topically.

U.S. Pat. No. 5,466,823 discloses a class of pyrazolyl benzene sulfonamide compoundscan be delivered intravascularly, intraperitoneally, subcutaneously, intramuscularly or topically.

U.S. Patent Application Publication No. 2003/0161867 discloses a dermally deliverable pharmaceutical composition comprises at least one selective cyclooxygenase-2 inhibitory drug or prodrug thereof solubilized in a pharmaceutically acceptable carrier that comprises a low molecular weight monohydric alcohol, and optionally a skin permeation enhancer.

However, none of the references disclose a hydro alcoholic clear topical gel formulation comprising one or more selective COX-2 inhibitors for therapeutic topical anti-inflammatory and analgesic action in a penetration vehicle system comprising a skin penetration enhancing effective amount of at least one monohydric alcohol and at least two non-volatile organic compounds selected from the group consisting of pyrrolidone, polyol ethers, polyols and mixtures thereof.

### SUMMARY OF THE INVENTION

It is a feature of the present invention to provide a therapeutic anti-inflammatory and/or analgesic topical hydroalcoholic clear gel pharmaceutical composition containing at least one or more selective COX-2 inhibitors or pharmaceutically acceptable salts or esters thereof for percutaneous delivery through the skin.

In accordance with one embodiment of the present invention, a hydroalcoholic topical pharmaceutical composition is provided comprising a therapeutically effective amount of a therapeutic agent comprising one or more selective cyclooxygenase-2 (COX-2) inhibitors or pharmaceutically acceptable salts or esters thereof solubilzed in a solubilizing amount of a penetration vehicle system comprising a skin penetration enhancing effective amount of at least one monohydric alcohol and at least two non-volatile organic compounds selected from the group consisting of pyrrolidones, polyol ethers, polyols and mixtures thereof.

In another embodiment of the present invention, a hydroalcoholic clear gel pharmaceutical composition is provided comprising:

(a) about 0.1 to about 10% of one or more selective COX-2 inhibitors or pharmaceutically acceptable salts or esters thereof;

(b) about 10% to about 90% of a skin penetration vehicle system comprising at least one monohydric alcohol and at least two non-volatile organic compounds selected from the group consisting of pyrrolidones, polyol ethers, polyols and mixtures thereof;

(c) about 0.05 to about 50% of a gelling agent;

(d) water q.s. to 100%; and optionally one or more of the following agents (e) about 0.005 to about 1% of an antioxidant;

(f) about 0.1 to about 20% of a counterirritant;

(g) about 0.0001 to about 0.01% of a coloring agent; and

(h) a neutralizing agent in an amount sufficient to adjust the pH of the composition in the range of about 3.0 to about 6.5, said percentages being w/w of the composition.

In another embodiment of the present invention, a process for preparing the therapeutic anti-inflammatory and/or analgesic topical hydroalcoholic clear gel pharmaceutical composition is provided comprising the following steps:

(a) mixing about 0.5 to about 65% w/w of at least one monohydric alcohol with about 0.5 to about 60% w/w of at least two nonvolatile organic compounds of a penetration enhancer selected from the group consisting of pyrrolidones and derivatives thereof, polyol ethers, polyols and mixtures thereof; and,

(b) adding to the mixture obtained in step (a) about 0.1 to about 10% w/w of one or more selective COX-2 inhibitors or pharmaceutically acceptable salts or esters thereof followed by stirring until the one or more selective COX-2 inhibitors are substantially dissolved to form a solution.

The process can further comprise the steps of

(c) adding about 0.005 to about 1% w/w of a antioxidant to the solution of step (b), followed by stirring until the antioxidant is substantially dissolved;

(d) adding water to the mixture of step (c) under stirring; and

(e) adding about 0.05 to about 50% w/w of a gelling agent to the mixture of step (d) under stirring (e.g., high shear stirring) and, optionally

(f) adding a neutralizing agent to the mixture of step (e) to adjust the pH of the composition in a range of about 3.0 to about 6.5 to obtain a substantially clear topical gel pharmaceutical composition.

In yet another embodiment of the present invention a method for delivering one or more selective COX-2 inhibitors or pharmaceutically acceptable salts or esters thereof such as valdecoxib through the skin in order to treat conditions situated beneath the skin is provided, the method comprising incorporating a therapeutically effective amount of a therapeutic agent comprising one or more cyclooxygenase-2 (“COX-2”) inhibitors or pharmaceutically acceptable salts or esters thereof into a skin penetration vehicle system comprising a skin penetration enhancing effective amount of at least one monohydric alcohol and at least two non-volatile organic compounds selected from the group consisting of pyrrolidones, polyol ethers, polyols and mixtures thereof to form a topical pharmaceutical composition and topically administering the topical pharmaceutical composition to the area of the skin of a patient in need of anti-inflammatory and/or analgesic action.

In still yet another embodiment of the present invention a method for providing increased analgesic and/or anti-inflammatory efficacy and speed of action of a therapeutic agent comprising a selective COX-2 inhibitor or pharmaceutically acceptable salts or esters thereof beneath the skin is provided comprising the step of topically administering topical pharmaceutical composition comprising a therapeutic agent comprising a therapeutically effective amount of one or more selective COX-2 inhibitors or pharmaceutically acceptable salts or esters thereof solubilized in a solubilizing amount of a penetration vehicle system comprising a skin penetration enhancing effective amount of a penetration vehicle base comprising at least one monohydric alcohol and at least two non-volatile organic compounds selected from the group consisting of pyrrolidones, polyol ethers, polyols and mixtures thereof to the skin of a patient.

One aspect of the present invention provides a topical hydroalcoholic clear gel composition of valdecoxib, a selective COX-2 inhibitor completely dissolved in a gel vehicle.

Another aspect of the present invention provides methods for the preparation of topical hydroalcoholic clear gels of valdecoxib comprising penetration enhancers.

A further aspect of the present invention provides methods for preparation of topical hydroalcoholic clear gel pharmaceutical dosage forms of valdecoxib, which are feasible on industrial scale.

Definitions

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host means causing a therapeutically effective blood concentration of the active ingredient at the particular location. This can be accomplished by, e.g., topical, local or by systemic administration of the active ingredient to the host.

By “pharmaceutically acceptable” is meant those salts and esters which are, within the scope of sound medical judgment, suitable for use in contact with the tissues of humans and lower animals without undue toxicity, irritation, allergic response and the like, commensurate with a reasonable benefit/risk ratio, and effective for their intended use. Representative acid additions salts include the hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarare, succinate, tartrate, ascorbate, glucoheptonate, lactobionate, lauryl sulphate salts and the like. Representative alkali or alkaline earth metal salts include the sodium, calcium, potassium and magnesium salts, and the like.

The term “subject” or “a patient” or “a host” as used herein refers to mammalian animals, preferably human.

### DETAILED DESCRIPTION OF THE INVENTION

The compositions of the present invention are intended for topical, non-invasive, application to the skin, particularly to the region where a therapeutic agent comprising one or more selective COX-2 inhibitor active ingredients or pharmaceutically acceptable salts or esters thereof is intended to exert its pharmacological activity, e.g., to a region of inflammation, injury or pain to the muscles or joints, or other form of cutaneous disorders or disruptions characterized by skin inflammation and/or hyperproliferative activity in the epidermis. Accordingly, the present invention provides a topical hydroalcoholic pharmaceutical composition comprising a therapeutically effective amount of a therapeutic agent comprising one or more selective COX-2 inhibitors or pharmaceutically acceptable salts or esters thereof solubilized in a solubilizing amount of a penetration vehicle system comprising a skin penetration enhancing effective amount of at least one monohydric alcohol and at least two non-volatile organic compounds selected from the group consisting of pyrrolidones, polyol ethers, polyols and mixtures thereof to deliver the therapeutic agent across the stratum corneum to the site of action. The present invention also provides a process of solubilization of the one or more selective COX-2 inhibitors in suitable penetration enhancing vehicle to provide a hydroalcoholic clear gel composition, which will enhance the flux of drug across the skin.

Any selective COX-2 inhibitor or pharmaceutically acceptable salts or esters thereof may be used in the topical formulations and methods of the present invention. Useful selective COX-2 inhibitors that can be used in this invention include, but are not limited to, those disclosed in U.S. Pat. Nos. 5,393,790; 5,418,254; 5,420,343; 5,466,823; 5,476,944; 5,486,534; 5,547,975; 5,565,482; 5,576,339; 5,580,985, 5,585,504; 5,593,994 and 5,596,008, the contents of which are incorporated by reference herein. More particularly, the useful COX-II inhibitors include the substituted spiro compounds of U.S. Pat. No. 5,393,790, e.g., 5-(4-fluorophenyl)-6-[4-(methylsulfonyl)phenyl]spiro[2.4]hept-5-ene, 4-[6-(4-fluorophenyl)spiro[2.4]hept-5-en-5-yl]benzenesulfonamide, 6-(4-fluorophenyl)-7-[4-(methylsulfonyl)phenyl]spiro[3.4]oct-6-ene, and the like; the sulfonamides of U.S. Pat. No. 5,409,944, e.g., 5-methanesulfonamido-6-(2-thienylthio)-1-indanone, 5-methanesulfonamido-6-(2-(4-methyl-1,3-diazinylthio))-1-indanone, 5-methanesulfonamido-6-(2-thiazolylthio)-1-indanone, and the like; the 2,3-substituted cyclopentadienyl compounds of U.S. Pat. No. 5,418,254, e.g., 1-methylsulfonyl-4-[1,1-dimethyl-4-(4-fluorophenyl)cyclopenta-2,4-dien-3-yl]benzene, 4-[4-(4-fluorophenyl)-1,1-dimethylcyclopenta-2,4-dien-3-yl]benzenesulfonamide, 1-methylsulfonyl-4-{4-(4-trifluoromethylphenyl)-1-trifluoromethylcyclopenta-2,4-dien-3-yl]benzene, and the like; the aromatic cycloethers of U.S. Pat. No. 5,420,343, e.g., methyl 3,5-bis(1,1-dimethylethyl)benzoate, 3,5-bis(1,1-dimethylethyl) benzenemethanol, 1,3-bis(1,1-dimethylethyl)-5-(2-chloroethyl)benzene, and the like; the 1-aroyl acids of U.S. Pat. No. 5,436,265, e.g., 1-(2,4,6-trichlorobenzoyl)-5-methoxy-2-methyl-3-indolyl acetic acid, 1-(2,6-dichlorobenzoyl)-5-methoxy-2-methyl-3-indolyl acetic acid and the like; the phenyl heterocycles of U.S. Pat. Nos. 5,474,995, 5,536,752, 5,550,142, 5,710,140 and 5,767,291, e.g., 3-(4-(aminosulfonyl)phenyl)-2-(4-fluorophenyl)thiophene, 2-(4-fluorophenyl)-3-(4-(methylsulfonyl)phenyl)-2-cyclopentenone, 4-(4-methylsulfonyl)phenyl)-5-(4-fluorophenyl)isothiazole, 4-(aminosulfonyl)phenyl)-2-(4-fluorophenyl)thiophene, 3-(4-(aminosulfonyl)phenyl)-2-4-(fluorophenyl)-5-(2-propyl)thiophene, 3-(4-(aminosulfonyl)phenyl)-2-cyclohexylthiophene, 3-(4-(aminosulfonyl)phenyl)-2-4-(fluorophenyl)-5-(2-hydroxy-2-propyl)thiophene, 3-(4-(aminosulfonyl)phenyl)-2-(4-fluorophenyl)thiophene and the like; the benzenesulfonamides of U.S. Pat. No. 5,466,823, e.g., 4-(5-(4-methylphenyl)-3-(trifluoromethyl)-1H-pyrazol-1-yl)benzenesulfonamide (which is also referred to as celecoxib) and the like; the cyclic phenolic thioether derivatives of U.S. Pat. No. 5,476,944, e.g., 3,5-bis(1,1-dimethylethyl)benzenethiol, trans-2-[[3,5-bis(1,1-dimethylethyl)henyl]hio]cyclohexanol, 3,6-dioxabicyclo-[3. 1.0]hexane, and the like; the 3,4-substituted pyrazoles of U.S. Pat. No. 5,486,534, e.g., 4-(4-fluorophenyl)-1-methyl-3-[4-(methylsulfonyl)phenyl]-5-trifluoromethyl)pyrazole, 1-benzyl-4-(4-fluorophenyl)-3-[4-(methylsulfonyl)phenyl]-5-(trifluoromethyl)pyrazole, 1-allyl-4(4-fluorophenyl)-3-[4-methylsulfonyl)phenyl]-5-(trifluoromethyl)-1H-pyrazole, and the like; the N-benzyl-3-indoleacetic acids of U.S. Pat. No. 5,510,368, e.g., 2-(5-bromo-1-(4-bromobenzyl)-2-methyl-1H-indol-3-yl)propionic acid, (S)-(+)-2-(5-bromo-1-(4-bromophenyl)-2-methyl-1H-indol-3-yl)acetyl acid, (R)-(−)-2-(5 -bromo-1-(4-bromobenzyl)-2-methyl-1H-indol-3-yl)propionic acid, and the like; the diaryl bicyclic heterocyclics of U.S. Pat. No. 5,521,213, e.g., 3-(4-(methylsulfonyl)phenyl)-2-phenylbenzo[b]furan, 3-(4-(methanesulfonyl)phenyl)-2-phenylbenzo[b]thiophene, 2-(4-fluorophenyl)-3-(4-aminosulfonyl)phenyl)-4H-thieno[2,3-c]furan-6-one, and the like; the benzopyranopyrazolyl derivatives of U.S. Pat. No. 5,547,975, e.g., 4-[1,4-dihydro-3-(trifluoromethyl)-[1]benzopyrano[4,3-c]pyrazol-1-yl]benzenesulfonamide, methyl [1-[4-(aminosulfonyl)phenyl]-1,4-dihydro-[1]benzopyrano[4,3-c]pyrazol-3-yl] carboxylate, 4-[3-(trifluoromethyl)-1H-benzofuro[3,2-c]pyrazol-1-yl] benzenesulfonamide, and the like; the aryl substituted 5,5 fused aromatic nitrogen compounds of U.S. Pat. No. 5,552,422, e.g., 5-(4-methylsulfonyl)phenyl)-6-phenylimidazo[2,1-b]thiazole, 2-methyl-5-(methylsulfonyl)phenyl)-6-phenylimidazo[2,1-b]thiazole, 3-methyl-5-(4-methylsulfonyl)phenyl)-6-phenylimidazo[2,1-b]thiazole, and the like; the heteroarylpyranopyrazolyl derivatives of U.S. Pat. No. 5,565,482, e.g., 4-[1,5-dihydro-6-fluoro-7-methoxy-3-(trifluoromethyl)-[2]benzothiopyrano[4,3-c]pyrazol, 4-[1,4-dihydro-3-(trifluoromethyl)-[1]benzopyrano-[4,3-c]pyrazol-1-yl]benzenesulfonamide, 1,5-dihydro-6-fluoro-7-methoxy-1-[(4-methylsulfonyl)phenyl]-3-(trifluoromethyl)-[2]benzothiopyrano-[4,3-c]pyrazol-1-yl]benzenesulfonamide, and the like; the pyridyl substituted cyclopentadienes of U.S. Pat. No. 5,576,339, e.g., 1-methylsulfonyl-4-[1,1-dimethyl-4-(4-fluorophenyl)cyclopenta-2,4-dien-3-yl]benzene, 4-[4-(4-fluoropyhenyl)-1,1-dimethylcyclopenta-2,4-dien-3-yl]benzenesulfonamide, and the like; the substituted pyrazoles of U.S. Pat. No. 5,580,985, e.g., 1-ethyl-4-(4-fluorophenyl)-3-[4-(methylsulfonyl)phenyl]-5-(trifluoromethyl)-1H-pyrazole, 3-amino-4,4,4-trifluoro-2(4-fluorophenyl)-1-[4-(methylthio)phenyl]-2-buten-1-one, 1-benzyl-4-(4-fluorophenyl)-3-[4-(methylsulfonyl)phenyl]-5-(trifluoromethyl)pyrazole, and the like; the lactones of U.S. Pat. No. 5,585,504, e.g., 3-phenyl-4-(4-methylsulfonyl)phenyl-2-(5H)-furanone, 3-(3,4-difluorophenyl)-4-(4-methylsulfonyl)phenyl)-2-(5H)-furanone, and the like; the ortho substituted phenyl compounds of U.S. Pat. No. 5,593,994, e.g., 2-[(4-methylthio)phenyl]-1-biphenyl, 1-cyclohexene-2-(4□-methylsulfonylphenyl) benzene, 3-(4□-methylsulfonylphenyl)-4-phenylphenol, and the like; the 3,4-diaryl substituted pyridines of U.S. Pat. No. 5,596,008, e.g., 5-(4-fluorophenyl)-2-methoxy-4-[4-(methylsulfonyl)phenyl]-6-(trifluoromethyl)pyridine, 2-ethoxy-5-(4-fluorophenyl)-4-[4-(methylsulfonyl)phenyl]-6-(trifluoromethyl)pyridine, 5-(4-fluorophenyl)-4-[4-(methylsulfonyl)phenyl]-2-(2-propynyloxy)-6-(trifluoromethyl)pyridine, and the like; the N-benzylindol-3-yl propanoic acid derivatives of U.S. Pat. No. 5,604,253, e.g., 3-[1-(p-Bromobenzyl)-5-methoxy-2-methylindol-3-yl]propanoic acid, 3-[1-(p-Bromobenzyl)-5-methoxy-2-methylindol-3-yl]-2,2-dimethyl-propanoic acid, 2-Benzyl-3-[1-(p-Bromobenzyl)-5-methoxy-2-methylindol-3-yl]propanoic acid and the like; the 5-methanesulfonamido-1-indanones of U.S. Pat. No. 5,604,260, e.g., 4-(2,4-Dichlorophenoxy)-3-nitrobenzaldehyde, 5-methanesulfonamido-6-(2,4-difluorophenylthio)-1-indanone and the like; the N-benzylindol-3-yl butanoic acid derivatives of U.S. Pat. No. 5,639,780, e.g., [4-(1-(4-Bromobenzyl)-5-methoxy-2-methyl-1-H-indol-3-yl)-3-(ethane-1,2-diyl)]butanoic acid, 4-(1-(4-Bromobenzyl)-5-methoxy-2-methyl-1-H-indol-3-yl)-2-methylbutanoic acid and the like; the diphenyl-1,2-3-thiadiazoles of U.S. Pat. No. 5,677,318, e.g., 4-Phenyl-5-(4-(methylsulfonyl)-phenyl-1,2,3-thiadiazole, 4-(4-fluorophenyl)-5-(4-(methylsulfonyl)phenyl-1,2,3-thiadiazole, 4-(3-fluorophenyl)-5-(4-(methylsulfonyl)phenyl-1,2,3-thiadiazole and the like; the diaryl-5-oxygenated-2-(5H)-furanones of U.S. Pat. No. 5,691,374, e.g., 5-hydroxy-3-(3,4-difluorophenyl)-4-(4-(methylsulfonyl)phenyl)-2-(5H)-furanone, 5-hydroxy-5-methyl-,4-(4-(methylsulfonyl)phenyl)-3-phenyl-2-(5H)-furanone, 5-hydroxy-4-(4-(methylsulfonyl)phenyl)-3-phenyl-2-(5H)-furanone and the like; the 3,4-diaryl-2-hydroxy-2,5-dihydrofuranes of U.S. Pat. No. 5,698,584, e.g., 3-(3,5-difluorophenyl)-5,5-dimethyl-2-hydroxy-4-(4-(methylsulfonyl)phenyl)-2,5-dihydrofuran, 5,5-dimethyl-3-(4-fluorophenyl)-2-hydroxy-4-(4-(methylsulfonyl)phenyl)-2,5-dihydrofuran, 5,5-dimethyl-2-ethoxy-3-(3-fluorophenyl)-4-(4-(methylsulfonyl)phenyl)-2,5-dihydrofuran and the like; the diphenyl stilbenes of U.S. Pat. No. 5,733,909, e.g., (E)-3-(4-methylsulfonyl)phenyl-2-phenylbut-2-enoic acid methyl ester, (E)-3-(methylsulfonyl)phenyl-2-phenylbut-2-enoic acid, (E)-3-(4-methylsulfonyl)phenyl-1-morpholin-4-yl-2-phenylbut-2en-1-one and the like; the alkylated styrenes of U.S. Pat. No. 5,789,413, e.g., 2-(3-fluorophenyl)-4-methyl-3-(4-(methylsulfonyl)-phenyl)-2-(Z)-penten-1.4-diol, acetic acid 4-acetoxy-2-(3-fluorophenyl)-4-methyl-3-(4-(methylsulfonyl)phenyl)-2-(Z)-pent-2-enyl ester, 2-(3-fluorophenyl)-4-methoxy-4-methyl-3-((4-methylsulfonyllphenyl)-2-(Z)-pentenoic acid and the like; the bisaryl cyclobutene derivatives of U.S. Pat. No. 5,817,700, e.g., 4,4-dichloro-3-(4-methylthiophenyl)-2-phenyl-2-cyclobuten-1-one, 4,4-dichloro-3-(4-methylsulfonylphenyl)-2-phenyl-2-cyclobuten-1-one, 4-chloro-3-(4-methylsulfonylphenyl)-2-phenyl-2-cyclobuten-1-one and the like, the contents of each of which are incorporated by reference herein. Other selective inhibitors of COX-II and methods of preparation thereof are set forth in, for example, WO99/30721, the contents of which are incorporated herein by reference.

In one embodiment of the present invention, the selective inhibitors of COX-2 for use herein include, but are not limited to, valdecoxib, celicoxib, paracoxib, etoricoxib, 4-[5-(4-methylphenyl)-3-(triluoromethyl)-1H-pyrazol-1-yl]benzenes-sulfonamide which is compound (4) of WO99/30721 and is denoted celicoxib (trade name Celebrex®) and 3-(phenyl)-4-(4-(methylsulfonyl)phenyl)-2-(5H)-furanone which is compound (63) of WO99/30721 and is denoted MK-0966 and Vioxx® or pharmaceutically acceptable salts or esters thereof. Another selective inhibitor of COX-2 is NS 398 which is N-(2-cyclohexyloxy-4-nitrophenyl) methanesulfonamide, commercially available from Cayman Chemical of Ann Arbor, Mich. In another embodiment of the present invention, the selective inhibitor of COX-2 for use herein is valdecoxib.

The dosage of the selective inhibitors of COX-2 for use in the compositions herein is a COX-2 inhibiting amount, which is a therapeutically effective amount and is an amount high enough to significantly positively modify the condition to be treated, but low enough to avoid serious side effects (at a reasonable benefit/risk ratio), within the scope of sound medical judgement. The precise amount of drug will vary with the specific drug, the ability of the composition to penetrate the drug through the skin, the amount of the composition to be applied, the particular condition being treated, the severity of the condition, the duration of the treatment, the nature of concurrent therapy, the age and physical condition of the patient being treated, and the like factors. The precise therapeutically effective amount of a COX-2 inhibitor to be used in the compositions and methods of the present invention can be determined by the ordinarily skilled artisan with consideration of such individual factors as, for example, age, weight, and condition of the patient. In one embodiment, the selective COX-2 inhibitors may be present in amount ranging from 0.1 to about 10% w/w.

The penetration of the selective COX-2 active ingredients through the skin is advantageously enhanced to an acceptable level by including in the composition a penetration vehicle system comprising a skin penetration enhancing effective amount of at least one monohydric alcohol and at least two non-volatile organic compounds selected from the group consisting of pyrrolidones, polyol ethers, polyols and mixtures thereof. Accordingly, the permeability of the drug through the skin is significantly improved.

Suitable monohydric alcohols for use herein include, but are not limited to, one or more alcohols having from 1 to about 20 carbon atoms. In another embodiment of the present invention, the suitable monohydric alcohols for use herein include, but are not limited to, one or more alcohols having from 2 to 6 carbon atoms. Exemplary alcohols for use herein include methanol, ethanol, isopropyl alcohol, butanol and the like and mixtures thereof. Preferably, the volatile organic solvent comprises ethanol. The use of one or more alcohols in the composition of the present invention advantageously acts as a skin penetration enhancer by rapidly evaporating when in contact with the skin and concentrating the active ingrediant in the residual formulation that remains on the skin. While not wishing to be bound by theory, it is believed that some thermodynamic activity will drive the drug into the stratum corneum. Additionally, the alcohol(s) can alter the physical integrity of the stratum corneum barrier resulting in an increase in the ability of the active ingrediant to penetrate the skin. Commercially, denatured alcohol such as SDA-40 can be used in place of Alcohol USP (ethanol) herein.

The non-volatile organic compounds for use herein include at least two non-volatile organic compounds selected from the group consisting of pyrrolidones, polyol ethers, polyols and mixtures thereof. Suitable pyrrolidones for use herein include, but are not limited to, vinyl pyrrolidones, 2-pyrrolidones, N-alkylpyrrolidones (e.g., N-methyl-2-pyrrolidones) and the like and mixtures thereof. A preferred pyrrolidone is N-methyl-2-pyrrolidone as a non-volatile organic compounds type of penetration enhancer. N-methyl-2-pyrrolidone is commercially available as Phannasolve® (available from ISP Corp. of Wayne, N.J.), and is a safe and compatible material. Pharmasolve® advantageously increases the water solubility of insoluble drugs and can therefore develop insoluble drugs into topical products.

The solubility enhancement can be attributed to three parameters: nonpolar molecular dispersion, polar type chemical bonding and hydrogen bonding. For example, N-methyl-2-pyrrolidone can undergo a large number of chemical reactions at various positions on the pyrrolidone ring. The enhanced solubility can be attributed to a complexing action with the nitrogen and carbonyl reactive centers of the molecule.

Pharmasolve® is a drug solubilizer and therefore enhances the bioavailability of topical formulations. In particular, it can increase the solubility of many drugs that are not water-soluble thereby enhancing their physico-chemical stability and bioavailability. Pharmasolve's® favorable safety profile makes it a solubilizer of choice for use in pharmaceutical formulations. Furthermore, Pharmasolve® has a favorable toxicity profile, making it a suitable candidate for use in a variety of topical dosage forms. Pharmasolve® is practically nonirritating to rabbit skin. (PII=0.5). According to the manufacturer's brochure, when Pharmasolve® was analyzed in a repeated insult patch test using 50 human subjects and a total of 15 applications, N-methyl-2-pyrrolidone was neither a primary dermal irritant nor a sensitizer.

Suitable polyol ethers for use herein include, but are not limited to, ethylene glycol monomethyl ether, ethylene glycol monoethyl ether, ethylene glycol monobutyl ether, ethylene glycol monopropyl ether, ethylene glycol monophenyl ether, ethylene glycol monohexyl ether, diethylene glycol monoethyl ether, diethylene glycol monomethyl ether, triethylene glycol monomethyl ether, triethylene glycol monoethyl ether, ethylene glycol monopropyl ether, ethylene glycol monobutyl ether, diethylene glycol monobutyl ether, triethylene glycol monobutyl ether, ethylene glycol monohexyl ether, diethyl glycol monohexyl ether, ethylene glycol phenyl ether, polypropylene glycol, polyethylene glycol, polyethylene glycol dodecyl ether, diethylene glycol monoethyl ether, polyethylene glycol-8-glyceryl caprylate and the like and mixtures thereof.

In one embodiment the polyol ether is diethylene glycol monoethyl ether, commercially available as Transcutol® (available from Gattefosse of St Priest Mi-Plaine, France). Transcutol® can solubilize hydrophobic materials. The increased drug flux across the stratum corneum can be achieved by the diffusion of Transcutol® into it, thereby changing the ability of the drug to penetrate the intercellular space. It is believed that the primary role of Transcutol® is the modification of the thermodynamic activity of the drug. By direct action on its solubility, Transcutol® favors the passage of larger quantities of the drug into the stratum corneum allowing a greater solubilization in the aqueous domains of the tissue. Primary Cutaneous Irritation test on rabbits concluded that Transcutol® is nonirritating. Cutaneous sensitization on humans using Marzulli and Maibach's method states that Transcutol® is nonirritating and non-sensitizing. Currently, there are various clinical studies conducted in the U.S. using Transcutol® D in topical products (Phase I, II and III clinical trials).

Suitable polyols for use herein include, but are not limited to, ethylene glycol, propylene glycol, butylene glycol, hexylene glycol, propylene glycol monocaprylate and mixtures thereof. A preferred polyol is propylene glycol. Propylene glycol is also used as a humectant in an amount of about 15%. See, e.g., A. Kibbe, Handbook of Pharmaceutical Excipients, 3rd Ed. (2000). In hydroalcoholic gel formulations, propylene glycol can assist in enhancing the flux of the drug across the skin once the alcohol evaporates by forming a saturated solution of the drug with the additional nonvolatile compound.

Accordingly, the penetration vehicle system for the selective COX-2 inhibitors is preferably an aqueous or non-aqueous alcoholic carrier containing sufficient monohydric alcohol, especially ethanol, and at least two of the foregoing non-volatile organic compounds to solubilize the selective COX-2 inhibitors and be miscible with the enhancers. In one embodiment, the at least two non-volatile organic solubilizing agents are at least one pyrrolidone and at least one polyol. In another embodiment, the at least two non-volatile organic compounds are N-methyl-2-pyrrolidone and propylene glycol. In another embodiment, the at least two non-volatile organic compounds are at least one pyrrolidone and at least one polyol ether and/or at least one polyol. In one embodiment, the amount of the penetration vehicle system will range from about 10 to about 90% w/w. In a second embodiment, the amount of the penetration vehicle system will range from about 50% to about 80% w/w. In preparing the penetration vehicle system, the monohydric alcohol will ordinarily be present in an amount ranging from about 0.5% to about 65% w/w in one embodiment. In a second embodiment, monohydric alcohol will be present in an amount ranging from about 30% to about 55% ww. In another embodiment, the monohydric alcohol will be present in a major amount in the composition, e.g., an amount of greater than or equal to 50%, based on the total weight of the composition. The at least two non-volatile organic compounds will ordinarily be present in an amount ranging from about 0.5% to about 60% w/w in one embodiment, from about 40% to about 60% w/w in a second embodiment and from about 27% to about 35% ww in a third embodiment.

Topical hydroalcoholic compositions of the present invention, to be useful for transdermal application to give systemic delivery of the drug, will ordinarily contain a selective COX-2 inhibitor (e.g., valdecoxib) in a concentration permitting a daily dosage amount of about 10 mg to about 100 mg, preferably about 20 mg to about 80 mg, for example about 30 mg to about 40 mg, illustratively about 32 mg to about 38 mg, more particularly about 34 mg to about 36 mg. Preferably the concentration is such that this dosage amount can be provided by application of the composition one to four times a day, preferably one to two times a day, to a skin area of up to about 400 cm², preferably about 1 cm² to about 100 cm².

Preferably, the topical compositions of the present invention are spreadable, semi-solid, jelly-like gels, which may be occluded (e.g., covered with a permeable, semi-permeable, or impermeable film or barrier) or remain unoccluded. However, the compositions of the present invention may also take the form of a spray, an aerosol, a lotion, a cream, or an ointment. The terms “gel”, “spray”, “aerosol”, “lotion”, “cream”, and “ointment”, are used herein as defined in Remington Pharmaceutical Sciences, 19th Edition, 1995, and are incorporated herein by reference.

The compositions of the present invention are generally formulated as gels, especially aqueous-alcoholic gels. However, other forms, such as, for example, lotions, creams, mousses, aerosols, ointments, lubricants, etc., may be used so long as when applied to the affected area of the skin the formulation will stay in place, i.e., without run-off, for sufficient time, to permit an individual to spread and retain the composition over and on the affected area. A suitable carrier system such as, for example, a thickening or gelling agent, is typically included to facilitate application of the formulation to the skin. If desired, an additional skin penetration enhancer such as dioxolane, dioxane or acetal can be included in the formulations in an amount effective to further enhance the penetration of the active selective COX-2 inhibitor ingredient through the skin, including the stratum corneum.

Suitable gelling agents for use in the pharmaceutical compositions of the present invention include, but are not limited to, synthetic or semi-synthetic polymeric materials, polyacrylate copolymers, cellulose derivatives, polymethyl vinyl ether/maleic anhydride copolymers and the like and mixtures thereof. Various grades of Carbopol such as, for example, Carbopol 934, 940, 941, 974, 980, 981, 1342, 5984, ETD2020, ETD 2050, Ultrez 10 (Manufacturer: Noveon) can be used in the present invention. Carbopols are a carbomer which are synthetic high molecular weight polymers of acrylic acid that are cross-linked with either allylsucrose or allylethers of pentaerythritol having a molecular weight of 3×10⁶. The gelation mechanism generally depends on neutralization of the carboxylic acid moiety to form a soluble salt. The polymer is hydrophilic and produces sparkling clear gels when neutralized. Carbomer gels possess good thermal stability in that gel viscosity and yield value are essentially unaffected by temperature. As a topical product, carbomer gels possess optimum rheological properties. The inherent pseudoplastic flow permits immediate recovery of viscosity when shear is terminated and the high yield value and quick break make it ideal for dispensing. In the pharmaceutical compositions of the present invention, carbomer gels are used as a suspending or viscosity-increasing agent. Aqueous solutions of Carbopol is acidic in nature due to the presence of free carboxylic acid residues. Neutralization of this solution cross-links and gelatinizes the polymer to form a viscous integral structure of desired viscosity. The gelling agent will ordinarily be present in an amount ranging from about 0.05 to about 50% w/w.

Suitable antioxidants for use in the pharmaceutical compositionof the present invention capable of reducing and/or preventing oxidative deterioration of the topical composition include, but are not limited to, butylated hydroxy toluene, butylated hydroxy anisole, alpha tocopherol, ascorbyl palmitate, butyl parahydroxybenzoate, methyl parahydroxybenzoate, propyl parahydroxybenzoate and the like and mixtures thereof. The antioxidants such as butylated hydroxy anisole have antimicrobial properties against, for example, molds and gram-positive bacteria. The antioxidants will ordinarily be present in an amount ranging from about 0.005 to about 1% w/w.

Counterirritants may also be used in the pharmaceutical compositions of the present invention which, when applied on the spot where it hurts, stimulate the cutaneous sensory receptors causing an inflammation or irritation of the skin. This hot, sometimes painful, burning sensation temporarily counters the original pain. Suitable counterirritants for use in the pharmaceutical composition of the present invention include, but are not limited to, methyl salicylate, capsaicin, menthol, camphor; turpentine oil and the like and mixtures thereof. An example of a counterirritant for use herein is methyl salicylate. On topical application, the counterirritant is absorbed through the skin and is applied for the relief of pain in rheumatic conditions and painful muscle or joints. The counterirritants will ordinarily be present in an amount ranging from about 0.1 to about 20% w/w.

Coloring agents for use in the compositions of the present invention may be selected from any colorant used in pharmaceuticals, which is approved and certified by the FDA. Coloring agents add aesthetic appeal to the topical preparation and will ordinarily be present in an amount ranging from about 0.0001 to about 0.01% w/w. A preferred coloring agent is Brilliant Blue FCF.

The compositions of the present invention may further contain a neutralizing agent to adjust the pH of the composition in the range of about 3.0 to about 6.5. Suitable neutralizing agents for use herein include, but are not limited to, organic basic compounds, inorganic basic compounds and the like and mixtures thereof. Suitable organic basic compounds include, but are not limited to, alkanolamines, e.g., methanolamine, ethanolamine, propanolamine, butanolamine, dimethanloamine, diethanolamine, dipropanolamine, dibutanolamine, diisopropanolamine, tributanolamine, aminomethylpropanol, N-methyl glucamine, tetrahydroxypropyl ethylene diamine and the like; alkylamines, e.g., methylamine, ethylamine, propylamine, butylamine, diethylamine, dipropylamine, isopropylamine and the like; and mixtures thereof. In one embodiment, the neutralizing agent is triethanolamine. Suitable inorganic basic compounds include, but are not limited to, ammonium hydroxide, alkali metal salts, alkaline earth metal salts such as magnesium oxide, magnesium hydroxide, calcium hydroxide, sodium hydroxide, potassium hydroxide, lithium hydroxide, aluminum hydroxide, potassium carbonate, sodium bicarbonate and the like and mixtures thereof.

If desired, the compositions of the present invention may further contain one or more pharmaceutically acceptable excipients. Suitable pharmaceutically acceptable excipients include, but are not limited to, diluents, retardants, lubricants and the like and mixtures thereof.

A particular feature of the present invention is that the dosage form can be designed so that the drug penetrates the skin to deliver a therapeutically effective amount of the drug to a target site such as, for example, epidermal, dermal, subcutaneous, muscular and articular organs and tissues, while maintaining systemic levels of the drug not greatly in excess of a minimum therapeutically effective level. Thus pharmaceutical compositions as described above can be used to effect targeted delivery of one or more selective COX-2 inhibitory drugs to an external or internal site of a COX-2 condition to be treated, e.g., pain and/or inflammation in a subject. According to a therapeutic method of the invention, a composition as provided herein is topically administered to a skin surface of the subject, preferably at a locus overlying or adjacent to the site of pain and/or inflammation.

Therapeutic methods and compositions of the invention are useful in treatment and prevention of a very wide range of disorders mediated by COX-2, including but not limited to, disorders characterized by inflammation, pain and/or fever. Such compositions are especially useful as anti-inflammatory agents, such as in treatment of arthritis, with the additional benefit of having significantly less harmful side effects than compositions of conventional non-selective non-steroidal anti-inflammatory drugs (NSAIDs) that lack selectivity for COX-2 over COX-1. In particular, the compositions of the present invention have reduced potential for gastrointestinal toxicity and gastrointestinal irritation including upper gastrointestinal ulceration and bleeding, reduced potential for renal side effects such as reduction in renal function leading to fluid retention and exacerbation of hypertension, reduced effect on bleeding times including inhibition of platelet function, and possibly a lessened ability to induce asthma attacks in aspirin-sensitive asthmatic subjects, by comparison with compositions of conventional NSAIDs. Thus, the compositions of the present invention are particularly useful as an alternative to conventional NSAIDs where such NSAIDs are contraindicated, for example in patients with peptic ulcers, gastritis, regional enteritis, ulcerative colitis, diverticulitis or with a recurrent history of gastrointestinal lesions; gastrointestinal bleeding, coagulation disorders including anemia such as hypoprothrombinemia, hemophilia or other bleeding problems; kidney disease; or in patients prior to surgery or patients taking anticoagulants.

Contemplated compositions are useful to treat a variety of arthritic disorders, including but not limited to rheumatoid arthritis, spondyloarthropathies, gouty arthritis, osteoarthritis, systemic lupus erythematosus and juvenile arthritis.

The compositions and processes of the present invention will now be illustrated by the following examples. The examples are not intended to be limiting of the scope of the present invention but read in conjunction with the detailed and general description above, provide further understanding of the present invention and an outline of a process for preparing the compositions of the invention.

### EXAMPLE 1

TABLE 1

| Ingredients/Components | Quantity (g) | % w/w |
|---|---|---|
| Valdecoxib (active ingredient) | 2.50 | 1.0 |
| N-methyl-2-pyrrolidone (non volatile | 18.75 | 7.5 |
| organic solvent type penetration | | |
| enhancer) | | |
| Ethanol (volatile organic solvent type | 125.0 | 50.0 |
| penetration enhancer) | | |
| Butylated Hydroxy Anisole | 0.25 | 0.1 |
| (antioxidant) | | |
| Propylene Glycol (non volatile organic | 25.00 | 10.0 |
| solvent type penetration | | |
| enhancer/humectant) | | |
| Carbopol 980 (gelling agent) | 3.75 | 1.5 |
| Water | 69.57 | 27.83 |
| Triethanolamine (alkalizing agent) | 0.175 | 0.07 |
| Methyl Salicylate (counterirritant) | 5.00 | 2.0 |
| Brilliant Blue FCF (coloring agent) | 0.00075 | 0.0003 |
| Total | 250.00 | |

Example 1 Processing Steps:
(a) Ethanol was mixed with N-methyl-2-pyrrolidone in a manufacturing vessel.
(b) Valdecoxib was added to the mixture of step (a) and stirred until completely dissolved.
(c) Butylated hydroxy anisole was added to the solution of step (b) with stirring until completely dissolved.
(d) Propylene glycol was added to the solution of step (c) with stirring.
(e) Water was added to the solution of step (d) with stirring.
(f) Carbopol 980 was added in small increments to the solution of step (e) with high shear mixing in a suitable mixer/homogenizer until a smooth hazy dispersion was obtained.
(g) Triethanolamine diluted with water was added to the dispersion of step (f) while stirring at slow speed with a paddle type or planetary type of stirrer under vacuum to produce a clear gel composition.
(h) Methyl Salicylate was added next with stirring under vacuum.
(i) Brilliant blue FCF was dissolved in water and added under vacuum to obtain a clear colored gel composition.

### EXAMPLE 2

TABLE 2

| Ingredients/Components | Quantity (g) | % w/w |
|---|---|---|
| Valdecoxib | 2.0 | 2.0 |
| Diethylene glycol monoethyl ether | 20.0 | 20.0 |
| Ethanol | 50.0 | 50.0 |
| Butylated Hydroxy Anisole | 0.1 | 0.1 |
| Propylene Glycol | 5.0 | 5.0 |
| Polyethylene Glycol-400 | 10.0 | 10.0 |
| Carbopol 980 | 1.5 | 1.5 |
| Water | 10.33 | 10.33 |
| Triethanolamine | 0.07 | 0.07 |
| Methyl Salicylate | 1.0 | 1.0 |
| Brilliant Blue FCF | 0.0003 | 0.0003 |
| Total | 100.0 | |

Example 2 Processing Steps:
(a) Ethanol was mixed with Diethylene glycol monoethyl ether in a manufacturing vessel.
(b) Valdecoxib was added to the mixture of step (a) and stirred until completely dissolved.
(c) Butylated hydroxy anisole was added to the solution of step (b) with stirring until completely dissolved.
(d) Propylene glycol and Polyethylene glycol-400 was added to the solution of step (c) with stirring.
(e) Water was added to the solution of step (d) with stirring.
(f) Carbopol 980 was added in small increments to the solution of step (e) with high shear mixing in a suitable mixer/homogenizer until a smooth hazy dispersion was obtained.
(g) Triethanolamine diluted with water was added to the dispersion of step (f) while stirring at slow speed with a paddle type or planetary type of stirrer under vacuum to produce a clear gel composition.
(h) Methyl Salicylate was added next with stirring under vacuum.
(i) Brilliant blue FCF was dissolved in water and added under vacuum to obtain a clear colored gel composition.

### EXAMPLE 3

### In vivo Anti-inflammatory Activity of Pharmaceutical Gel for Topical Delivery is given in Table 3

The study was conducted on male Wistar rats weighing 150-180 gm. The valdecoxib gel formulations 0.5%, 1% and 2% w/w of the present invention were compared with the conventional gel formulations, Diclofenac gel 1% w/w (Voveran Emulgel™, Novartis), Nimesulide gel 1% w/w (Nimulid Transgel™, Panacea Biotech) and Rofecoxib gel 1% w/w (Rofibax gel™, Ranbaxy Labs). For each formulation, animals were divided into control and four test groups [n=6]. The rat paw volumes were measured initially to obtain basal reading using Plethysmometer (Ugo Basile, Italy). An aliquot of the specified test formulation was weighed according to the dose and was applied on the left hind paw of the rat topically. The formulation was rubbed 25 times with finger on the sub plantar region of the left hind paw. Control group animals received similar treatment (by weighing same amount) with placebo gel on the left hind paw. Three hours after the treatment, 0.1 ml of 1% w/v carrageenan (Sigma Chemicals, USA) in saline was injected in the sub plantar region of the left hind paw for both test and control group animals. Paw volumes were measured for all rats at 3 hours after the challenge of carrageenan. The anti-inflammatory activity of the valdecoxib gel formulation for different test groups were expressed as percent inhibition of paw volume of rats in the control group. The results of the study with different gel formulations at different dose levels are tabulated below:

TABLE 3

| | Percent inhibition of edema formation at dose | | | |
|---|---|---|---|---|
| | (mg/Kg) of active drug | | | |
| Study | 1 mg/Kg | 3 mg/Kg | 10 mg/Kg | 30 mg/Kg |
| Valdecoxib gel | 25.32 ± 2.49 | 41.83 ± 4.64 | 46.50 ± 4.52 | 53.48 ± 4.32 |
| 0.5% w/w | | | | |
| Valdecoxib gel | 33.67 ± 4.11 | 45.83 ± 2.96 | 51.33 ± 3.57 | 52.38 ± 1.44 |
| 1% w/w | | | | |
| Valdecoxib gel | 41.50 ± 3.48 | 47.38 ± 4.38 | 44.57 ± 3.94 | 61.02 ± 3.99 |
| 2% w/w | | | | |
| Diclofenac gel | 28.20 ± 1.50 | 34.95 ± 4.01 | 40.12 ± 4.37 | 42.41 ± 4.01 |
| 1% w/w | | | | |
| (Voveran | | | | |
| Emulgel ™) | | | | |
| Nimesulide gel | 30.32 ± 4.26 | 52.50 ± 4.68 | 51.67 ± 5.00 | 53.63 ± 2.26 |
| 1% w/w | | | | |
| (Nimulid | | | | |
| Transgel ™) | | | | |
| Rofecoxib gel | 34.83 ± 3.42 | 35.97 ± 2.36 | 40.50 ± 4.96 | 52.67 ± 4.29 |
| 1% w/w | | | | |
| (Rofibax | | | | |
| gel ™) | | | | |

The results of the in vivo data indicated that the anti-inflammatory activity of valdecoxib gels 0.5%, 1% and 2% of the present invention was comparable with that of the other commercial preparations.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims

1. A hydroalcoholic topical pharmaceutical composition comprising a therapeutically effective amount of a therapeutic agent comprising one or more selective cyclooxygenase-2 (COX-2) inhibitors or pharmaceutically acceptable salts or esters thereof solubilzed in a solubilizing amount of a penetration vehicle system comprising a skin penetration enhancing effective amount of at least one monohydric alcohol and at least two non-volatile organic compounds selected from the group consisting of pyrrolidones, polyol ethers, polyols and mixtures thereof.

2. The composition of claim 1, wherein substantially all of the therapeutic agent present is in solubilized form.

3. The composition of claim 1, wherein the selective COX-2 inhibitor or pharmaceutically acceptable salts or esters thereof is selected from the group consisting of valdecoxib, celicoxib, paracoxib, etoricoxib, MK-0966, NS 398 and mixtures thereof.

4. The composition of claim 1, wherein the selective COX-2 inhibitor or pharmaceutically acceptable salts or esters thereof is valdecoxib or a prodrug thereof.

5. The composition of claim 1, wherein the monohydric alcohol is a C₂ to C₆ monohydric alcohol.

6. The composition of claim 1, wherein the monohydric alcohol is selected from the group consisting of ethanol, isopropanol and mixtures thereof.

7. The composition of claim 1, wherein the at least two non-volatile organic compounds are at least one pyrrolidone and at least one polyol.

8. The composition of claim 7, wherein the pyrrolidone is N-methyl-2-pyrrolidone and the polyol is selected from the group consisting of ethylene glycol, propylene glycol, butylene glycol, hexylene glycol, propylene glycol monocaprylate and mixtures thereof.

9. The composition of claim 1, wherein the at least two non-volatile organic compounds are at least one pyrrolidone and at least one polyol ether and at least one polyol.

10. The composition of claim 1, wherein the polyol ether is selected from the group consisting of ethylene glycol monomethyl ether, ethylene glycol monoethyl ether, ethylene glycol monobutyl ether, ethylene glycol monopropyl ether, ethylene glycol monophenyl ether, ethylene glycol monohexyl ether, diethylene glycol monoethyl ether, diethylene glycol monomethyl ether, triethylene glycol monomethyl ether, triethylene glycol monoethyl ether, ethylene glycol monopropyl ether, ethylene glycol monobutyl ether, diethylene glycol monobutyl ether, triethylene glycol monobutyl ether, ethylene glycol monohexyl ether, diethyl glycol monohexyl ether, ethylene glycol phenyl ether, polypropylene glycol, polyethylene glycol, polyethylene glycol dodecyl ether, diethylene glycol monoethyl ether, polyethylene glycol-8-glyceryl caprylate and mixtures and the polyol is selected from the group consisting of ethylene glycol, propylene glycol, butylene glycol, hexylene glycol, propylene glycol monocaprylate and mixtures thereof.

11. The composition of claim 1, which is in a liquid or semi-solid dosage form.

12. The composition of claim 11, in a dosage form selected from the group consisting of creams, pastes, gels, ointments, lotions and aerosols.

13. The composition of claim 1, further comprising a gelling agent.

14. The composition of claim 13, further comprising at least one compound selected from the group consisting of an antioxidant, a counterirritant and a neutralizing agent to adjust the pH of the composition in the range of about 3.0 to about 6.5.

15. The composition of claim 1, wherein the selective COX-2 inhibitor or pharmaceutically acceptable salts or esters thereof is valdecoxib or a prodrug thereof, the monohydric alcohol is selected from the group consisting of ethanol, isopropanol and mixtures thereof and the at least two non-volatile organic compounds are at least one pyrrolidone and at least one polyol.

16. The composition of claim 15, wherein the pyrrolidone is N-methyl-2-pyrrolidone and the polyol is selected from the group consisting of ethylene glycol, propylene glycol, butylene glycol, hexylene glycol, propylene glycol monocaprylate and mixtures thereof.

17. The composition of claim 1, wherein the selective COX-2 inhibitor or pharmaceutically acceptable salts or esters thereof is valdecoxib or a prodrug thereof, the monohydric alcohol is selected from the group consisting of ethanol, isopropanol and mixtures thereof and the at least two non-volatile organic compounds are a polyol ether and a polyol.

18. The composition of claim 17, wherein the polyol ether is selected from the group consisting of ethylene glycol monomethyl ether, ethylene glycol monoethyl ether, ethylene glycol monobutyl ether, ethylene glycol monopropyl ether, ethylene glycol monophenyl ether, ethylene glycol monohexyl ether, diethylene glycol monoethyl ether, diethylene glycol monomethyl ether, triethylene glycol monomethyl ether, triethylene glycol monoethyl ether, ethylene glycol monopropyl ether, ethylene glycol monobutyl ether, diethylene glycol monobutyl ether, triethylene glycol monobutyl ether, ethylene glycol monohexyl ether, diethyl glycol monohexyl ether, ethylene glycol phenyl ether, polypropylene glycol, polyethylene glycol, polyethylene glycol dodecyl ether, diethylene glycol monoethyl ether, polyethylene glycol-8-glyceryl caprylate and mixtures and the polyol is selected from the group consisting of ethylene glycol, propylene glycol, butylene glycol, hexylene glycol, propylene glycol monocaprylate and mixtures thereof.

19. The composition of claim 1, wherein the selective COX-2 inhibitor is present in an amount of about 0.1% to about 10% w/w.

20. The composition of claim 1, wherein the penetration vehicle system is present in the range of about 10% to about 90% w/w.

21. A process for the preparation of a therapeutic anti-inflammatory and analgesic clear gel composition for topical use, comprising:
(a) mixing about 0.5 to about 65% w/w of at least one monohydric alcohol with about 0.5 to about 60% w/w of at least two nonvolatile organic compounds of a penetration enhancer selected from the group consisting of pyrrolidones and derivatives thereof, polyol ethers, polyols and mixtures thereof, and
(b) adding to the mixture obtained in step (a) about 0.1 to about 10% w/w of one or more selective COX-2 inhibitors or pharmaceutically acceptable salts or esters thereof followed by stirring until the one or more selective COX-2 inhibitors are substantially dissolved to form a solution.

22. The process of claim 21, further comprising
(c) adding water to the mixture of step (b); and
(d) adding about 0.05 to about 50% w/w of a gelling agent to the mixture of step (c).

23. The process of claim 22, further comprising
(e) adding about 0.005 to about 1% w/w of a antioxidant to the solution of step (d); and
(f) adding a neutralizing agent to the mixture of step (e) to adjust the pH of the composition in a range of about 3.0 to about 6.5 to obtain a clear gel.

24. The process of claim 21, wherein the selective COX-2 inhibitor or pharmaceutically acceptable salts or esters thereof is selected from the group consisting of valdecoxib, celicoxib, paracoxib, etoricoxib, MK-0966, NS 398 and mixtures thereof.

25. The process of claim 21, wherein the selective COX-2 inhibitor or pharmaceutically acceptable salts or esters thereof is valdecoxib or a prodrug thereof.

26. The process of claim 21, wherein the monohydric alcohol is a C₂ to C₆ monohydric alcohol.

27. The process of claim 21, wherein the monohydric alcohol is selected from the group consisting of ethanol, isopropanol and mixtures thereof.

28. The process of claim 21, wherein the at least two non-volatile organic compounds are at least one pyrrolidone and at least one polyol.

29. The process of claim 28, wherein the pyrrolidone is N-methyl-2-pyrrolidone and the polyol is selected from the group consisting of ethylene glycol, propylene glycol, butylene glycol, hexylene glycol, propylene glycol monocaprylate and mixtures thereof.

30. The process of claim 21, wherein the at least two non-volatile organic compounds are at least one polyol ether and at least one polyol.

31. The process of claim 30, wherein the polyol ether is selected from the group consisting of ethylene glycol monomethyl ether, ethylene glycol monoethyl ether, ethylene glycol monobutyl ether, ethylene glycol monopropyl ether, ethylene glycol monophenyl ether, ethylene glycol monohexyl ether, diethylene glycol monoethyl ether, diethylene glycol monomethyl ether, triethylene glycol monomethyl ether, triethylene glycol monoethyl ether, ethylene glycol monopropyl ether, ethylene glycol monobutyl ether, diethylene glycol monobutyl ether, triethylene glycol monobutyl ether, ethylene glycol monohexyl ether, diethyl glycol monohexyl ether, ethylene glycol phenyl ether, polypropylene glycol, polyethylene glycol, polyethylene glycol dodecyl ether, diethylene glycol monoethyl ether, polyethylene glycol-8-glyceryl caprylate and mixtures and the polyol is selected from the group consisting of ethylene glycol, propylene glycol, butylene glycol, hexylene glycol, propylene glycol monocaprylate and mixtures thereof.

32. A topical gel pharmaceutical composition comprising:
(a) about 0.1% to about 10% w/w of a therapeutic agent comprising one or more selective COX-2 inhibitors or pharmaceutically acceptable salts or esters thereof;
(b) about 10% to about 90% w/w of a penetration vehicle system comprising a skin penetration enhancing effective amount of at least one monohydric alcohol and at least two non-volatile organic compounds selected from the group consisting of pyrrolidones, polyol ethers, polyols and mixtures thereof; and
(c) about 0.05% to about 50% w/w of a gelling agent.

33. The composition of claim 32, wherein the at least one monohydric alcohol is present in an amount of about 0.5% to about 65% w/w.

34. The composition of claim 32 wherein the at least two non-volatile organic compounds are present in an amount of about 0.5% to about 60% w/w.

35. A method of effecting targeted delivery of a selective COX-2 inhibitor to a site of pain and/or inflammation in a subject, the method comprising topically administering the composition of claim 1 to skin of the subject.

36. The method of claim 35, wherein the composition is administered to skin at a locus overlying or adjacent to the site of pain and/or inflammation.

37. The method of claim 35, wherein the site of pain and/or inflammation is in an epidermal, dermal, subcutaneous, muscular or articular tissue.

38. A method of effecting targeted delivery of a selective COX-2 inhibitor to a site of pain and/or inflammation in a subject, the method comprising topically administering the composition of claim 15 to skin of the subject.

39. A method of effecting targeted delivery of a selective COX-2 inhibitor to a site of pain and/or inflammation in a subject, the method comprising topically administering the composition of claim 17 to skin of the subject.
